(19) United States
(12) Patent Application Publication
(10) Pub. No.: US 2014/0323878 A1
(43) Pub. Date: Oct. 30, 2014

(54) SCANNING PROBE, SCANNING OBSERVATION SYSTEM, INTEGRATED ENDOSCOPE, AND INTEGRATED ENDOSCOPE SYSTEM

(71) Applicant: HOYA CORPORATION, Tokyo (JP)

(72) Inventors: Shunsuke Toriumi, Tokyo (JP); Shotaro Kobayashi, Tokyo (JP)

(21) Appl. No.: 14/363,475

(22) PCT Filed: Jan. 11, 2013
(86) PCT No.: PCT/JP2013/050414

§ 371 Date: Jun. 6, 2014

(30) Foreign Application Priority Data
Feb. 3, 2012 (JP) 2012-022229

(51) Int. Cl. A61B 1/00; A61B 5/00; A61B 1/12

(52) U.S. Cl. CPC A61B 1/00172; A61B 1/00165; A61B 1/128; A61B 5/0062; A61B 5/0084
USPC 600/478

(57) ABSTRACT

An optical fiber that transmits scanning light to a subject; a housing holding the optical fiber; a vibration element fixed to an exit end part of the fiber and vibrating periodically the exit end part so that the scanning light emitted from the exit end part scans on the subject in a predetermined trajectory; a hollow tube formed so an inner circumferential surface of the hollow tube positioned outside a moving range of the exit end part and the vibration element surrounds an entire circumference of the exit end part and a movable part of the vibration element; a thermal detection sensor detecting temperature in a hollow space of the hollow tube; and a heating element laid on one of the inner and outer circumferential surface of the hollow tube and increases the temperature in the hollow space in response to the temperature detected by the thermal detection sensor.

### TECHNICAL FIELD

The present invention relates to a scanning probe which scans on a subject with scanning light emitted from a light source, a scanning observation system including the scanning probe, an integrated endoscope integrally provided with the scanning probe and an electronic endoscope. and an integrated endoscope system including the integrated endoscope.

### BACKGROUND ART

A scanning observation system which images a subject, such as a living tissue, with a scanning probe is known. The scanning probe of this type scans on a subject by emitting scanning light from an exit end of an optical fiber while vibrating the optical fiber using, for example, a piezoelectric actuator. A concrete configuration of a scanning probe is described, for example, in a domestic re-publication of PCT international application No. 2010-503890A (hereafter, referred to as “patent document 1”; see, for example, FIG. 8).

The scanning probe described in patent document 1 has a cylindrical housing in which an optical fiber is accommodated and held in a state of a cantilever, and scans on a subject by periodically vibrating a free end of the optical fiber while applying a voltage to a piezoelectric tube. The scanning probe includes a thermal sensing device and a heater, and controls the heater based on the temperature sensed by the thermal sensing device so that the temperature in the housing is kept constant. As a result, effect to the piezoelectric tube having a thermal property (change of thermal expansion coefficient or piezo effect which depends on temperature change), and thereby it becomes possible to prevent a problem where a scanned image deforms depending on operating environment.

### SUMMARY OF THE INVENTION

It is considered that the housing provided in the scanning probe described in patent document 1 is a metal component having a high degree of accuracy of dimension because various optical components including a lens and the optical fiber need to be held in a state where axes of the optical components coincide with each other. However, in general metal has a high degree of thermal conductivity. Therefore, when the heater provided on an inner wall surface of the housing is heated, a large amount of heat is radiated to the outside of the housing. Therefore, it is difficult in the configuration described in patent document 1 to keep the temperature in the housing constant.

The present invention is made in consideration of the above described circumstances. That is, the object of the present invention is to provide a scanning probe, a scanning observation system, an integrated endoscope and an integrated endoscope system suitable for reducing deformation of a scanned image which depends on the temperature change by keeping temperature in a housing constant.

According to an embodiment of the invention, there is provided a scanning probe scanning on a subject with scanning light emitted from a light source, comprising: an optical fiber that transmits the scanning light to the subject; a housing that accommodates and holds the optical fiber; a vibration element that is fixed to an exit end part of the optical fiber and vibrates periodically the exit end part so that the scanning light emitted from the exit end part of the optical fiber scans on the subject in a predetermined trajectory; a hollow tube formed such that an inner circumferential surface of the hollow tube positioned outside a moving range of the exit end part and the vibration element surrounds an entire circumference of the exit end part and at least a movable part of the vibration element; a thermal detection sensor that detects temperature in a hollow space of the hollow tube; and a heating element that is laid on one of the inner circumferential surface and an outer circumferential surface of the hollow tube and is configured to increase the temperature in the hollow space in response to the temperature detected by the thermal detection sensor, and wherein a heat radiation resistance is disposed between the outer circumferential surface of the hollow tube and an inner wall surface of the housing.

According to the scanning probe of the embodiment of the invention, thanks to the heat radiation resistance provided between the outer circumferential surface of the hollow tube and an inner wall surface of the housing, heat of the heating element becomes hard to be radiated to the outside of the housing. Therefore, the temperature in the hollow space of the hollow tube, i.e., the temperature of a portion surrounding at least a movable part of the vibration element, can be easily kept at constant. Consequently, the scanning probe is suitable for reducing deformation of a scanned image by suppressing change of the property of the vibration element which depends on the temperature change.

The heat radiation resistance is one of an air layer provided between the outer circumferential surface of the hollow tube and the inner wall surface of the housing, and a thermal insulation material provided between the outer circumferential surface of the hollow tube and the inner wall surface of the housing.

The scanning probe according to an embodiment of the invention is configured such that the heating element is laid on the outer circumferential surface of the hollow tube. In this case, it is preferable that the hollow tube is a metal member so as to effectively radiate the heat of the heating element to the hollow space in a state where unevenness of heat distribution is reduced.

The scanning probe according to an embodiment of the invention is configured such that the heating element is laid on the inner circumferential surface of the hollow tube. In this case, it is preferable that the hollow tube is a resin molded component so that the heat of the heating element becomes hard to be radiated to the outside of the hollow space.

A scanning observation system according to an embodiment of the invention comprises: the above described scanning probe; and a heating element control means that controls the heating element so as to keep the temperature in the hollow space based on the temperature detected by the thermal detection sensor at a predetermined constant temperature.

An integrated endoscope according to an embodiment of the invention is integrally provided with an electronic endoscope in which a solid state image pick-up device for imaging a subject through an objective optical system is installed, and the above described scanning probe. In the integrated endoscope, a thermal insulation tape is wound around an outer wall surface of the housing; and the solid state image pick-up device and the housing on which the thermal insulation tape is wound are accommodated and held in a tip part of the integrated endoscope such that the solid state image pick-up device and the housing are parallel with each other.

An integrated endoscope system according to an embodiment of the invention comprises: the above described integrated endoscope; and a heating element control means that controls the heating element so as to keep the temperature in the hollow space based on the temperature detected by the thermal detection sensor at a predetermined constant temperature.

According to the invention, a scanning probe, a scanning observation system, an integrated endoscope and an integrated endoscope system suitable for reducing deformation of a scanned image which depends on the temperature change by keeping the temperature in the housing constant.

### BRIEF DESCRIPTION OF THE DRAWINGS

FIG. 1 is a block diagram illustrating a configuration of an endoscope system according to an embodiment of the invention.

FIG. 2 is a cross sectional view illustrating an internal structure of a confocal optical unit provided in an integrated endoscope according to the embodiment of the invention.

FIG. 3 is a cross sectional view illustrating an internal structure of a confocal optical unit provided in an integrated endoscope according to a variation of the embodiment of the invention.

FIG. 4 is a cross sectional view illustrating an internal structure of a confocal optical unit provided in an integrated endoscope according to another embodiment of the invention.

### EMBODIMENTS FOR CARRYING OUT THE INVENTION

In the following, an endoscope system according to an embodiment of the present invention is explained with reference to the accompanying drawings.

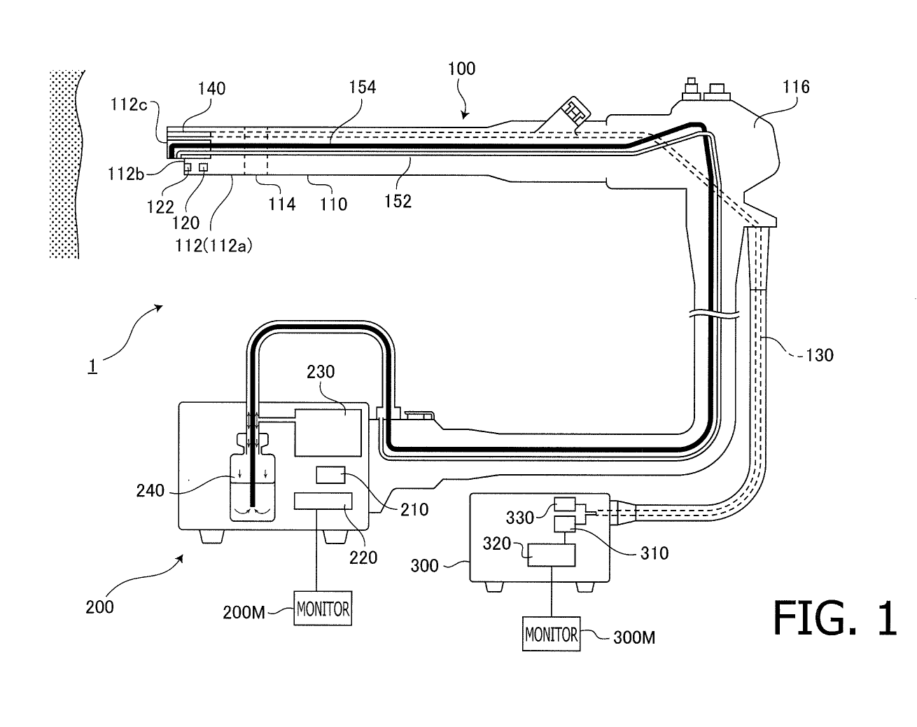

FIG. 1 is a block diagram illustrating a configuration of an endoscope system 1 according to the embodiment. As shown in FIG. 1, the endoscope system 1 has an integrated endoscope 100 for imaging a subject. The integrated endoscope 100 includes an insertion unit elastic tube 110 which is covered with an elastic sheath. To a tip of the insertion unit elastic tube 110, a proximal end of a tip part 112 covered with a resin housing (hereafter referred to as a “tip exterior housing 112a”) having rigidity is connected. A bending part 114 located at a joint part of the insertion unit elastic tube 110 and the tip part 112 is configured to be able to bend freely through a remote operation from a near-side operation unit 116 connected to a proximal end of the insertion unit elastic tube 110. This bending mechanism is a known mechanism installed in a general electronic scope, and is configured to bend the bending part 114 by a drawing motion of operation wires operated in conjunction with a rotation operation of a bending operation knob of the near-side operation unit 116. An imaging area of the integrated endoscope 100 moves as the direction of the tip part 112 changes depending on the bending motion caused by the rotation operation to the bending operation knob.

In the endoscope system 1, two imaging systems are installed. One is an imaging system (hereafter, referred to as a “normal imaging system”) similar to a general endoscopic imaging system which images a subject at normal magnification and resolution. Another is an imaging system (hereafter, referred to as a “confocal imaging system”) which images a subject at magnification and resolution higher than those of the normal imaging system.

The endoscope system 1 includes an electronic endoscope processor 200 constituting the normal imaging system. As shown in FIG. 1, the electronic endoscope processor 200 includes a light source 210 and an image processing controller 220. The light source 210 includes, for example, an igniter, a lamp and a dimmer mechanism, and is coupled to an entrance end of an LCB (Light Carrying Bundle; not shown) provided throughout the length of the integrated endoscope 100 (i.e., from the joint part with the electronic endoscope processor 200 to the tip exterior housing 112a). The illumination light which has entered the entrance end of the LCB propagates through the LCB, and exits from an exit end of the LCB provided in the tip exterior housing 112a.

The tip end surface of the tip exterior housing 112a has a first surface 112b and a second surface 112c which are formed to have a step therebetween. On the first surface 112b, a pair of light distribution lenses (not shown) and an electronic endoscope objective lens 122 which constitute the normal imaging system are disposed. The illumination light exiting from the exit end of the LCB illuminates the subject through the light distribution lenses. The light reflected from the subject forms, through the electronic endoscope objective lens 122, an optical image on a light-receiving surface of a solid state image pick-up device 120 mounted in the tip exterior housing 112a.

The solid state image pick-up device 120 is, for example, a single-chip color CCD (Charge Coupled Device) image sensor having a bayer image pixel array, and is configured to be driven at timings synchronizing with a video frame rate, in accordance with clock pulses supplied from the image processing controller 220. The solid state image pick-up device 120 accumulates charges corresponding to the light amount of the optical image formed on pixels on the light-receiving surface and converts the optical image into an image signal corresponding to respective colors of R, G and B. The converted image signal is inputted to the image processing controller 220 via a signal cable (not shown), subjected to predetermined image processing, and converted into a video signal, such as, NTSC (National Television System Committee) or PAL (Phase Alternating Line) complying with a predetermined standard. The converted video signal is sequentially inputted to a monitor 200M, and thereby a color image of the subject is displayed on the monitor 200M at normal magnification and resolution.

The electronic endoscope processor 200 includes an air pump 230 and a liquid tank 240. When a predetermined operation is made to the near-side operation unit 116, pressurized air is supplied from the air pump 230 to an air supply pipe 152. The pressurized air flows through the air supply pipe 152 toward the tip part 112 side, and is discharged to the outside from an air vent which is exposed from the tip exterior housing 112a. Further, when a predetermined another operation is made to the near-side operation unit 116, pressurized air is supplied from the air pump 230 to the liquid tank 240. The liquid (washing water) pressurized in the liquid tank 240 flows through a liquid supply pipe 154 toward the tip 112 side, and is discharged from a liquid vent which is exposed from the tip exterior housing 112a. The fluid discharged from the air vent or the liquid vent washes the first surface 112b (in particular, the electronic endoscope objective lens 122) of the tip exterior housing 112a. Thus, the image obtained by the normal imaging system is made clear. It should be noted that the air pump 230 and the liquid tank 240 are not the essential components constituting the normal imaging system. The air pump 230 and the liquid tank 240 are not necessarily provided in the electronic endoscope processor 200, and may be provided in a separate device which is separately provided from the electronic endoscope processor 200.

The endoscope system 1 includes a confocal processor 300 which constitutes the confocal imaging system. Confocal observation through the confocal imaging system is performed in a state where the second surface 112c of the tip exterior housing 112a is butted against the subject so as to obtain an image at a vertical layer position from a surface layer, such as biological mucosa of the subject, to a deep layer. On the other hand, when normal observation is performed using the normal imaging system, an arrangement plane (i.e., the first surface 112b) of the electronic endoscope objective lens 122 needs to be moved away from the subject by a distance corresponding to, for example, the focal length of the electronic endoscope objective lens 122. For this reason, the tip exterior housing 112a is configured such that the second surface 112c protrudes by a predetermined length with respect to the first surface 112b. Therefore, when the second surface 112c is butted against the subject, the electronic endoscope objective lens 122 stationarily stops at a position where the subject is within the depth of field.

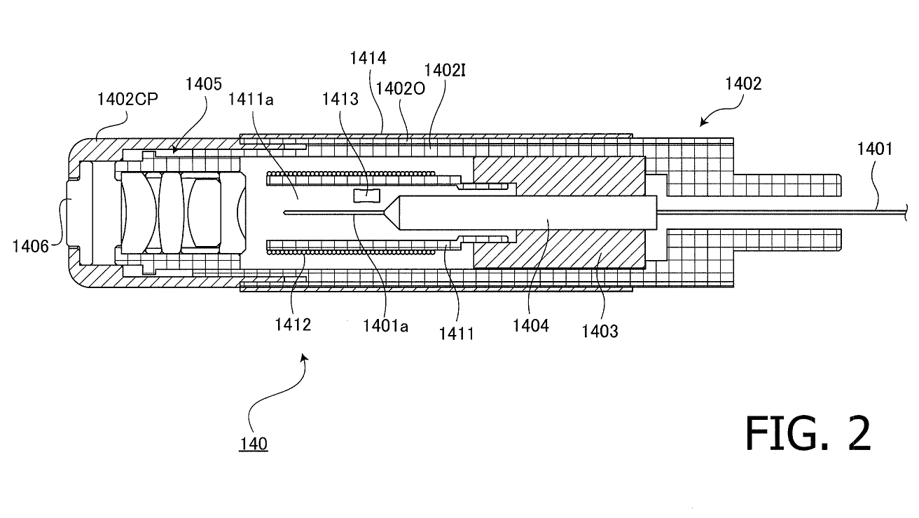

As shown in FIG. 1, the confocal processor 300 includes a confocal light source 310, an image processing controller 320 and a thermal controller 330. In the integrated endoscope 100, a scanning probe 130 is installed throughout the approximately entire length of the integrated endoscope 100 (from the joint part with the confocal processor 300 to the tip exterior housing 112a). In the tip exterior housing 112a, a tip part (a confocal optical unit 140) of the scanning probe 130 is accommodated and held to be parallel with the solid state image pick-up device 120. The confocal optical unit 140 and the confocal light source 310 have a known optical configuration designed to be adapted for the endoscope system 1 while utilizing the principal of a confocal microscope, and are optically coupled via a confocal optical fiber 1401 (FIG. 2).

FIG. 2 is a cross sectional view illustrating an internal structure of the confocal optical unit 140. As shown in FIG. 2, the confocal optical unit 140 is armored with a housing 1402 which accommodates various components. The housing 1402 includes an inner tube 1402I and an outer tube 1402O which are made of metal. The inner tube 1402I is accommodated in the outer tube 1402O to be slidable in an axial direction with respect to the outer tube 1402O and to be coaxial with the outer tube 1402O, and holds a mount member 1403 and an objective optical unit 1405. The mount member 1403 and the objective optical unit 1405 slide together with the inner tube 1402I in the axial direction with respect to the outer tube 1402O.

The mount member 1403 is a resin member having a ring-shape, and is configured such that a root of a piezoelectric tube 1404 is fitted into a ring-shaped hollow part of the mount member 1403. The piezoelectric tube 1404 holds the tip part of the confocal optical fiber 1401, for example, by adhesion. The objective optical unit 1405 is configured such that an objective optical system having a plurality of lenses are held by a lens holding frame. At the tip of the confocal optical unit 140, a cap 1402CP which seals the accommodation space in the housing 1402 and holds a cover glass 1406 is disposed. The cap 1402CP is adhered and fixed to the outer tube 1402O, and is slidable together with the outer tube 1402O in the axial direction with respect to the inner tube 1402I.

The confocal light source 310 emits laser light having the wavelength serving as excitation light for the subject and lets the laser light enter the proximal end (the entrance end) of the confocal optical fiber 1401. The excitation light propagates through the confocal optical fiber 1401, and exits from the exit end of an exit end part 1401a of the confocal optical fiber 1401. Further, the piezoelectric tube 1404 vibrates periodically in accordance with the voltage applied from the image processing controller 320 via wiring (not shown). Thus, the exit end of the exit end part 1401a functions as a secondary point source of the confocal imaging system which moves periodically to draw a predetermined trajectory in a substantially flat plane which is approximately perpendicular to the axial direction. Therefore, the excitation light emitted from the exit end of the exit end part 1401a two-dimensionally scans on the subject via the objective optical unit 1405 and the cover glass 1406. Further, it is possible to let the excitation light emitted from the exit end of the exit end part 1401a three-dimensionally scan on the subject, by changing the distance between the second surface 112c (the cover glass 1406 butted against the subject) and the exit end of the exit end part 1401a while sliding the inner tube 1402I and the outer tube 1402O using, for example, a Z-axis actuator (not shown).

Since the exit end of the exit end part 1401a of the confocal optical fiber 1401 is positioned at an image side focal point of the objective optical system of the objective optical unit 1405, the exit end functions as a confocal pinhole. That is, of the fluorescence emitted from the subject being illuminated with the excitation light, only florescence emitted from a convergence point optically conjugate with the exit end enters the exit end of the exit end part 1401a. The fluorescence which has entered the exit end of the exit end part 1401a is transmitted to the confocal light source 310, and is separated and detected with respect to the excitation light emitted from the laser source, and then is inputted to the image processing controller 320.

The image processing controller 320 executes sampling and holding at a constant rate for a detection signal and executes A-D conversion to obtain a digital detection signal. For an image corresponding to the direction of the above described substantially flat plate, assigning to a pixel address of a point image represented by the digital detection signal is performed in accordance with a predetermined remapping table in which signal detection timing and a pixel position (pixel address) are associated with each other, and consequently a two-dimensional image is generated. Further, the position of the point source (the exit end of the exit end part 1401a of the confocal optical fiber 1401) in the axial direction is constantly monitored in the confocal optical unit 140, and is transferred to the image processing controller 320. The image processing controller 320 generates a two-dimensional image for each of the positions in the axial direction, with reference to the information of the position of the point source in the axial direction. By compositing the two-dimensional images respectively corresponding to the positions in the axial direction, a three-dimensional image is obtained. The image processing controller 320 converts the signal of the generated three-dimensional image to a video signal complying with a predetermined standard, such as NTSC or PAL, and outputs the video signal to the monitor 300M. Thus, a three-dimensional confocal image of the subject is displayed on the display screen of the monitor 300M at high magnification and resolution.

The outer shape of the tip exterior housing 112a is formed such that a part of the tip exterior housing 112a including the second surface 112c which protrudes toward the object side with respect to the first surface 112b is situated within the angle of view of the electronic endoscope objective lens 122. Therefore, on the display screen of the monitor 200M, a part of the tip exterior housing 112a accommodating the confocal optical unit 140 is displayed in addition to the image of the subject represented at normal magnification and resolution. As a result, an operator is able to recognize the positional relationship between the subject and the second surface 112c through the display screen of the monitor 200M.

In the meantime, the temperature in the inner tube 1402I is unstable if no measures are taken. For example, the temperature in the inner tube 1402I is decreased by cool air of the pressurized fluid flowing through the air supply pipe 152 or the liquid supply pipe 154, increased by heating of the electric components, such as the solid state image pick-up device 120, or fluctuates by the effect of the temperature of the internal body into which the integrated endoscope 100 is inserted. When the thermal expansion coefficient and the piezo effect of the piezoelectric tube 1404 are changed depending on such temperature changes, the moving trajectory of the point source (the exit end of the exit end part 1401a of the confocal optical fiber 1401) changes, and thereby a degree of deformation of the scanned image increases. For this reason, in the embodiment, the confocal optical unit 140 is configured as follows.

Specifically, the confocal optical unit 140 includes a hollow tube 1411, a heater 1412, a thermal detection sensor 1413 and a thermal insulation tape 1414. The hollow tube 1411 is a metal component having a cylindrical shape, and is adhered and fixed to the mount member 1403. The hollow tube 1411 accommodates the exit end part 1401a of the confocal optical fiber 1401 and at least a movable part (a part which vibrates by application of a voltage) of the piezoelectric tube 1404 in a hollow space 1411a defined by an inner circumferential surface of the hollow tube 1411. More specifically, the inner circumferential surface of the hollow tube 1411 surrounds the entire circumference of the exit end part 1401a and at least the movable part of the piezoelectric tube 1404 outside a moving range of the exit end part 1401a and the piezoelectric tube 1404 so as to avoid mechanical interference between the hollow tube 1411 and the exit end part 1401a and the piezoelectric tube 1404.

The heater 1412 and the thermal detection sensor 1413 are connected with the thermal controller 330 via wiring (not shown). The heater 1412 is an electric resistance heater, such as a coil resistance heater, a thin film resistor heater or a cartridge resistance heater, and is uniformly provided substantially on the entire outer circumferential surface of the hollow tube 1411. The thermal detection sensor 1413 is, for example, a thermoelectric couple, a resistive thermal device or a thermistor, and is provided at the position where the temperature in the hollow space 1411a can be detected (in the example shown in FIG. 2, the thermal detection sensor 1413 is adhered and fixed to the inner circumferential surface of the hollow tube 1411).

The thermal controller 330 controls the heater 1412 based on the temperature detected by the thermal detection sensor 1413 so as to keep the temperature in the hollow space 1411a constant via the hollow tube 1411. The temperature in the hollow space 1411a is kept at a temperature (e.g., 42° C. to 43° C.) which is higher than, for example, the internal body temperature and does not affect the living body. It should be noted that the temperature control range in the hollow space 1411a is appropriately set depending on the imaging purpose and use of the confocal imaging system or the physical property of each component constituting the confocal optical unit 140.

If heat of the heater 1412 is radiated in a large amount to the outside of the inner tube 1402I, it becomes difficult to keep the temperature in the hollow space 1411a constant. By contrast, according to the embodiment, the whole circumference (space between the outer circumferential surface of the hollow tube 1411 and the inner wall surface of the inner tube 1402I) of the hollow tube 1411 is surrounded by an air layer. Since the air layer functions as a high heat radiation resistance, the heat of the heater 1412 is hard to be radiated to the outside of the inner tube 1402I. Therefore, in the confocal optical unit 140 according to the embodiment, it is easily to keep the temperature in the hollow space 1411a constant. Therefore, the confocal optical unit 140 suitably suppresses deformation of the scanned image by suppressing change of the property of the piezoelectric tube 1404 which depends on the temperature change. In addition, since heat of the heater 1412 transmits though the hollow tube 1411 once, the heat is radiated to the hollow space 1411a in a state where unevenness of heat distribution peculiar to the heater 1412 is reduced. As a result, it becomes possible to more suitably suppress change of the property of the piezoelectric tube 1404 which depends on the temperature change, and thereby it becomes possible to further suppress deformation of the scanned image.

On the outer circumferential surface of the outer tube 1402O, the resin tape 1414 having the thermal insulation performance is wound. The confocal optical unit 140 is positioned by being adhered to the wall in the tip exterior housing 112a with the resin tape 1414. The inside of the inner tube 1402I becomes hard to be affected by the effect of cool air of pressurized fluid flowing through the air supply pipe 152 or the liquid supply pipe 154, heating of the electronic components such as the solid state image pick-up device 120, and the internal body temperature. Therefore, temperature change in the inside of the inner tune 1402I is further suppressed.

The foregoing is the explanations about the embodiment of the invention. It is understood that the present invention is not limited to the above described embodiment, and can be varied in various ways within the technical scope of the invention. For example, in the embodiment, the scanning probe 130 and the electronic scope are formed as an integrated device (the integrated endoscope 100); however, in another embodiment the scanning probe 130 and the electronic scope may be configured as separate devices. In this case, the scanning probe 130 is used, for example, in a state where the scanning probe 130 is inserted into a forceps channel provided in the electronic scope.

In the above described embodiment, the scanning probe 130 and the confocal processor 300 constituting the confocal optical system are explained; however in another embodiment the scanning probe 130 and the confocal processor 300 may be replaced with a full color type scanning fiber endoscope and a corresponding processor (a scanning endoscope system described, for example, in patent document 1 in which a color subject image can be obtained through a scanning fiber as an alternative to the solid state image pickup device such as CCD).

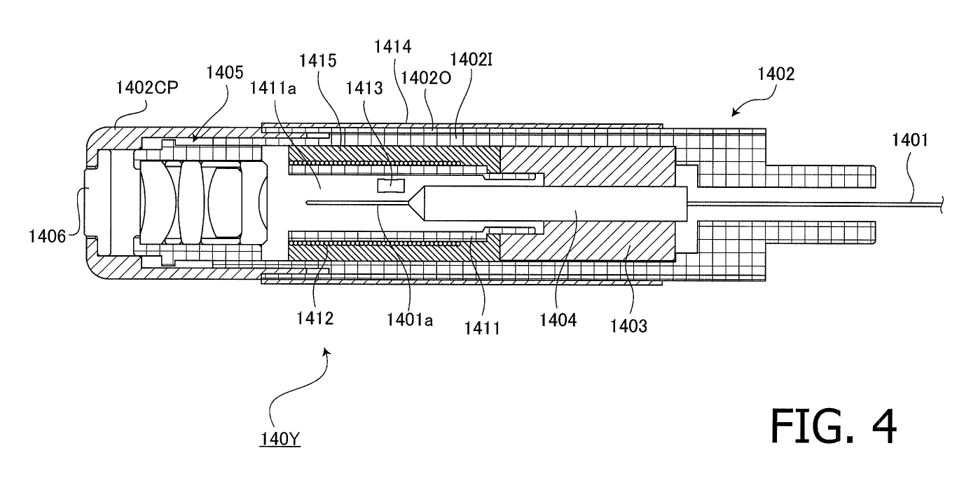

FIG. 4 is a cross sectional view illustrating an internal configuration of a confocal optical unit 140Y according to another embodiment. In FIG. 4, to elements which are the same as or similar to the confocal optical unit 140 shown in FIG. 2, the same or similar reference numbers are assigned, and explanations thereof are simplified or omitted. In the above described embodiment, an air layer surrounding the entire circumference of the hollow tube 1411 is used as heat radiation resistance; however, in another embodiment shown in FIG. 4, space between the outer circumferential surface of the hollow tube 1411 and the inner wall surface of the inner tube 1402I may be filled with thermal insulation material 1415 in place of the air layer.

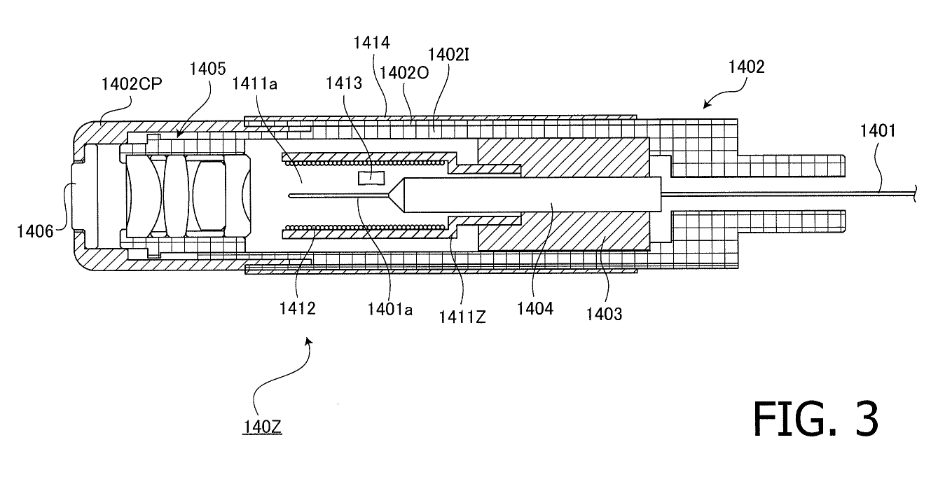

FIG. 3 is a cross sectional view illustrating an internal configuration of a variation (a confocal optical unit 140Z) of the confocal optical unit 140 according to the above described embodiment. In FIG. 3, to elements which are the same as or similar to the confocal optical unit 140 shown in FIG. 2, the same or similar reference numbers are assigned, and explanations thereof are simplified or omitted.

As shown in FIG. 3, the confocal optical unit 140Z according to the variation includes a hollow tube 1411Z. The hollow tube 1411Z is, for example, a resin molded component, and is provided with the heater 1412 over the substantially entire inner circumferential surface of the hollow tube 1411Z. The thermal detection sensor 1413 is adhered and fixed to the inner circumferential surface of the hollow tube 1411Z in an area where the heater 1412 is not provided. Since, in the variation, heat of the heater 1412 is radiated directly to the hollow space 1411a, temperature control for the hollow space 1411a becomes easier. Further, since the hollow tube 1411Z is formed as a resin molded component having a low degree of thermal conductivity, heat radiated from the heater 1412 becomes hard to be radiated to the outside of the hollow space 1411a. As a result, the heat loss during the temperature control for the hollow space 1411a can be suppressed, and thereby it becomes possible to suppress the amount of heat to be produced by the heater 1412.

1. A scanning probe scanning on a subject with scanning light emitted from a light source, comprising:
an optical fiber that transmits the scanning light to the subject;
a housing that accommodates and holds the optical fiber;
a vibration element that is fixed to an exit end part of the optical fiber and vibrates periodically the exit end part so that the scanning light emitted from the exit end part of the optical fiber scans on the subject in a predetermined trajectory;
a hollow tube formed such that an inner circumferential surface of the hollow tube positioned outside a moving range of the exit end part and the vibration element surrounds an entire circumference of the exit end part and at least a movable part of the vibration element;
a thermal detection sensor that detects temperature in a hollow space of the hollow tube; and
a heating element that is laid on one of the inner circumferential surface and an outer circumferential surface of the hollow tube and is configured to increase the temperature in the hollow space in response to the temperature detected by the thermal detection sensor,
wherein a heat radiation resistance is disposed between the outer circumferential surface of the hollow tube and an inner wall surface of the housing.

2. The scanning probe according to claim 1,
wherein the heat radiation resistance is one of an air layer provided between the outer circumferential surface of the hollow tube and the inner wall surface of the housing, and a thermal insulation material provided between the outer circumferential surface of the hollow tube and the inner wall surface of the housing.

3. The scanning probe according to claim 1,
wherein:
the heating element is laid on the outer circumferential surface of the hollow tube; and
the hollow tube is a metal member.

4. The scanning probe according to claim 1,
wherein:
the heating element is laid on the inner circumferential surface of the hollow tube; and
the hollow tube is a resin molded component.

5. A scanning observation system, comprising:
a scanning probe according to claim 1; and
a heating element controller that controls the heating element so as to keep the temperature in the hollow space based on the temperature detected by the thermal detection sensor at a predetermined constant temperature.

6. An integrated endoscope, comprising:
an electronic endoscope in which a solid state image pick-up device for imaging a subject through an objective optical system is installed; and
a scanning probe according to claim 1,
wherein the electronic endoscope and the scanning probe are integrally provided in the integrated endoscope, and
wherein:
a thermal insulation tape is wound around an outer wall surface of the housing; and
the solid state image pick-up device and the housing on which the thermal insulation tape is wound are accommodated and held in a tip part of the integrated endoscope such that the solid state image pick-up device and the housing are parallel with each other.

7. An integrated endoscope system, comprising:
an integrated endoscope according to claim 6; and
a heating element controller that controls the heating element so as to keep the temperature in the hollow space based on the temperature detected by the thermal detection sensor at a predetermined constant temperature.
